(19) United States
(12) Patent Application Publication
(10) Pub. No.: US 2003/0027799 A1
(43) Pub. Date: Feb. 6, 2003

(54) Methods of treating cancer using a combination of drugs

(75) Inventor: Jacques Jolivet, Laval (CA)

Correspondence Address: MILLEN, WHITE, ZELANO & BRANIGAN, PC, 2200 CLARENDON BLVD, SUITE 1400, ARLINGTON, VA, 22201, US

(73) Assignee: Shire BioChem Inc., Laval (CA)

(21) Appl. No.: 10/107,795

(22) Filed: Mar. 28, 2002

Related U.S. Application Data
(60) Provisional application No. 60/279,770, filed on Mar. 30, 2001.

(51) Int. Cl. A61K 31/675; A61K 31/513

(52) U.S. Cl. 514/87; 514/88; 514/267

(57) ABSTRACT

The present invention provides a novel method for treating a patient with cancer comprising administering to the patient a therapeutically effective amount of cisplatin and a compound having the formula I:

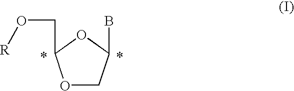

wherein B is cytosine or 5-fluorocytosine and R is selected from the group comprising H, monophosphate, diphosphate, triphosphate, carbonyl substituted with a C₁₋₆ alkyl, C₂₋₆ alkenyl, C₂₋₆ alkynyl, C₆₋₁₀ aryl, and

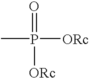

wherein each Rc is independently selected from the group comprising H, C₁₋₆ alkyl, C₂₋₆ alkenyl, C₂₋₆ alkynyl and a hydroxy protecting group.

This application claims the benefit of U.S. Provisional Application Serial No. 60/279,770 filed Mar. 30, 2001 which is hereby incorporated in its entirety.

### FIELD OF THE INVENTION

The present invention relates to pharmaceutical combinations and to methods useful in the treatment of cancer. Particularly, the combinations of this invention relate to dioxolane nucleosides in combination with cisplatin.

### BACKGROUND OF THE INVENTION

Cancer is the second leading cause of death in the United States. It is estimated that cancer is responsible for 30% of all deaths in the Western world. Lung, colorectal, breast and prostate cancers are the four biggest killers.

Many nucleoside analogues have been found to possess anticancer activity. It was reported in (Weitman et al Clinical Cancer Research (2000), 6(4), pp 1574-1578 and Giles et al Journal of Clinical Oncology (2001), 19(3), pp 762-771 and also Gourdeau et al Cancer Chemother. Pharmacol. (2001), 47(3), pp 236-240) that roxacitabine (β-L-dioxolane cytidine, β-L-OddC, Troxatyl™), a nucleoside analogue, has shown to have potent activity in the treatment of various forms of cancers (e.g. solid tumours, adult leukemia and lymphomas).

Cisplatin has been used in the treatment of refractory lymphomas, usually in combination with cytosine arabinoside and high-dose dexamethasone, as reported in Hematology, 2^{nd} Edition, edited by Ronald Hoffman et al., 1995. Despite improvements in the outcome of patients with current combination treatment programs, there exists a need to find other combination of drugs which exhibit antitumor activity.

The present invention provides a combination of troxacitabine with cisplatin which exhibits antitumor activity and would greatly aid in the development of new combination therapy against cancer.

### SUMMARY OF THE INVENTION

In one aspect, there is provided a novel pharmaceutical combination useful for the treatment of cancer in a mammal comprising a therapeutically effective amount of cisplatin and at least one active compound of formula I:

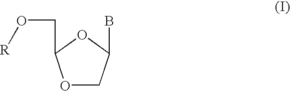

or a pharmaceutically acceptable salt thereof,

wherein B is cytosine or 5-fluorocytosine and R is selected from the group comprising H, monophosphate, diphosphate, triphosphate, carbonyl substituted with a C₁₋₆ alkyl, C₂₋₆ alkenyl, C₂₋₆ alkynyl, C₆₋₁₀ aryl and

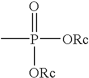

wherein each Rc is independently selected from the group comprising H, C₁₋₆ alkyl, C₂₋₆ alkenyl, C₂₋₆ alkynyl and a hydroxy protecting group.

In another aspect, there is provided a novel pharmaceutical combination useful for the treatment of cancer in a mammal comprising a therapeutically effective amount of cisplatin and at least one compound according to formula I and at least one further therapeutic agent chosen from the group comprising chemotherapeutic agent, multi-drug resistance reversing agent and biological response modifier.

The pharmaceutical combinations of the present invention are useful in cancer therapy, in particular in the treatment of cancer selected from the group comprising lung cancer, prostate cancer, bladder cancer, colorectal cancer, pancreatic cancer, gastric cancer, breast cancer, ovarian cancer, soft tissue sarcoma, osteosarcoma, hepatocellular carcinoma, leukemia and lymphomas in patients.

In another aspect, the pharmaceutical combinations of the present invention are useful in cancer therapy, in particular in the treatment of cancer selected from the group comprising colorectal cancer, melanoma, gastric cancer, islet cell cancer of the pancreas, non-small cell lung cancer (NSCLC), renal cancer, cervical cancer, breast cancer, ovarian cancer, squamous cell cancer of the pelvis, liver cancer, abdominal cancer and penile cancer.

In another aspect, there is provided a novel method of treating a patient having cancer comprising administering to said patient a therapeutically effective amount of cisplatin and at least one active compound of formula I.

In another aspect, there is provided a method of treating a patient having cancer comprising administering to said patient a therapeutically effective amount of cisplatin and at least one compound according to formula I and at least one further therapeutic agent chosen from the group comprising chemotherapeutic agent, multi-drug resistance reversing agent and biological response modifier.

In another aspect, there is provided a method of treating a patient having cancer, in particular in the treatment of cancer selected from the group comprising lung cancer, prostate cancer, bladder cancer, colorectal cancer, pancreatic cancer, gastric cancer, breast cancer, ovarian cancer, soft tissue sarcoma, osteosarcoma, hepatocellular carcinoma, leukemia and lymphomas, comprising administering to said patient a therapeutically effective amount of cisplatin and at least one compound according to formula I.

In another aspect, there is provided a method of treating a patient having cancer, in particular in the treatment of cancer selected from the group comprising colorectal cancer, melanoma, gastric cancer, islet cell cancer of the pancreas, non-small cell lung cancer (NSCLC), renal cancer, cervical cancer, breast cancer, ovarian cancer, squamous cell cancer of the pelvis, liver cancer, abdominal cancer and penile cancer.

In another aspect, there is provided a pharmaceutical composition for treating cancer in a patient comprising cisplatin, at least one compound according to formula I and at least one pharmaceutically acceptable carrier or excipient.

In another aspect, there is provided a pharmaceutical composition for treating cancer comprising cisplatin and at least one compound according to formula I and at least one further therapeutic agent.

In another aspect, there is provided a pharmaceutical composition for treating cancer comprising cisplatin, at least one compound according to formula I and at least one further therapeutic agent selected from the group comprising chemotherapeutic agents; multi-drug resistance reversing agents; and biological response modifiers.

In another aspect of the invention is the use of at least one compound according to formula I and cisplatin for the manufacture of a medicament for treating cancer in a mammal.

### DETAILED DESCRIPTION OF THE INVENTION

The present invention provides a novel pharmaceutical combination useful for the treatment of cancer in a mammal comprising a therapeutically effective amount of cisplatin and a compound having formula I:

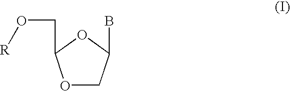

or a pharmaceutically acceptable salt thereof,

wherein B is cytosine or 5-fluorocytosine and R is selected from the group comprising H, monophosphate, diphosphate, triphosphate, carbonyl substituted with a C₁₋₆ alkyl, C₂₋₆ alkenyl, C₂₋₆ alkynyl, C₆₋₁₀ aryl, and

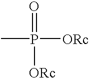

wherein each Rc is independently selected from the group comprising H, C₁₋₆ alkyl, C₂₋₆ alkenyl, C₂₋₆ alkynyl and a hydroxy protecting group.

In one embodiment, R is H.

In one embodiment, B is cytosine.

In one embodiment, B is 5-fluorocytosine.

In one embodiment, a compound of formula I is (−)-β-L-Dioxolane-Cytidine (β-L-OddC).

In one embodiment, a compound of formula I is (−)-β-Dioxolane-5-fluoro-Cytidine (5-FddC).

In one embodiment, the compounds of formula I of the present invention are provided substantially in the form of the (−) enantiomer. By “substantially” is meant that there is more of the (−) enantiomer than the (+) enantiomer.

In another embodiment, the compounds of formula I of the present invention are at least 95% free of the corresponding (+) enantiomer.

In another embodiment, the compounds of formula I of the present invention are at least 97% free of the corresponding (+) enantiomer.

Still in another embodiment, the compounds of formula I of the present invention are at least 99% free of the corresponding (+) enantiomer.

It will be appreciated by those skilled in the art that the compounds of formula (I) contain at least two chiral centres. The compounds of formula (I) thus exist in the form of two different optical isomers (i.e. (+) or (−) enantiomers or β-L and β-D). All such enantiomers and mixtures thereof including racemic mixtures are included within the scope of the invention. The single optical isomer or enantiomer can be obtained by method well known in the art, such as chiral HPLC, enzymatic resolution and the use of chiral auxiliary. Alternatively, the enantiomers of the compounds of formula (I) can be synthesized by using optically active starting materials.

In another embodiment, the pharmaceutical combinations of the present invention are useful in cancer therapy, in particular in the treatment of cancer selected from the group comprising lung cancer, prostate cancer, bladder cancer, colorectal cancer, pancreatic cancer, gastric cancer, breast cancer, ovarian cancer, soft tissue sarcoma, osteosarcoma, hepatocellular carcinoma, leukemia and lymphomas in patients.

In another embodiment, the pharmaceutical combinations of the present invention are useful in cancer therapy, in particular in the treatment of cancer selected from the group comprising colorectal cancer, melanoma, gastric cancer, islet cell cancer of the pancreas, non-small cell lung cancer (NSCLC), renal cancer, cervical cancer, breast cancer, ovarian cancer, squamous cell cancer of the pelvis, liver cancer, abdominal cancer and penile cancer.

In one embodiment, there is provided a novel pharmaceutical combination useful for the treatment of cancer in a mammal comprising a therapeutically effective amount of cisplatin and a compound having formula I wherein the compound of formula I is β-L-OddC.

In one embodiment, there is provided a novel pharmaceutical combination useful for the treatment of cancer in a mammal comprising a therapeutically effective amount of cisplatin and at least one compound according to formula I and at least one further therapeutic agent chosen from the group comprising chemotherapeutic agent, multi-drug resistance reversing agent and biological response modifier.

In one embodiment, the further therapeutic agent is a chemotherapeutic agent.

In one embodiment, the further therapeutic agent is a chemotherapeutic agent chosen from Asparaginase, Bleomycin, Busulfan, Carmustine, Chlorambucil, Cladribine, Cyclophosphamide, Cytarabine, Dacarbazine, Daunorubicin, Doxorubicin, Etoposide, Fludarabine, Gemcitabine, Hydroxyurea, Idarubicin, Ifosfamide, Lomustine, Mechlorethamine, Melphalan, Mercaptopurine, Methotrexate, Mitomycin, Mitoxantrone, Pentostatin, Procarbazine, 6-Thioguanine, Topotecan, Vinblastine, Vincristine, Dexamethasone, Retinoic acid and Prednisone.

In another embodiment, the further therapeutic agent is a chemotherapeutic agent chosen from Cytarabine, Etoposide, Mitoxantron, Cyclophosphamide, Retinoic acid, Daunorubicin, Doxorubicin and Idarubicin.

In another embodiment, the further therapeutic agent is Doxorubicin.

In one embodiment, the further therapeutic agent is a multi-drug resistance reversing agent.

In one embodiment, the further therapeutic agent is PSC 833.

In one embodiment, the further therapeutic agent is a biological response modifier.

In one embodiment, the further therapeutic agent is a biological response modifier chosen from monoclonal antibodies and cytokines.

In another embodiment, the further therapeutic agent is a cytokine chosen from interferons, interleukins and colony-stimulating factors.

In another embodiment, the further therapeutic agent is a biological response modifier chosen from Rituxan, CMA-676, Interferon-alpha recombinant, Interleukin-2, Interleukin-3, Erythropoetin, Epoetin, G-CSF, GM-CSF, Filgrastim, Sargramostim and Thrombopoietin.

In another embodiment, the individual components of such combinations as defined above may be administered either sequentially or simultaneously in separate or combined pharmaceutical formulations.

The combinations referred to above may conveniently be presented for use in the form of a pharmaceutical formulation and thus pharmaceutical formulations comprising a combination as defined above together with a pharmaceutically acceptable carrier therefore comprise a further aspect of the invention.

In another aspect, the present invention provides a novel method of treating a patient having cancer comprising administering to said patient a therapeutically effective amount of cisplatin and a compound having formula I:

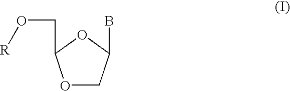

or a pharmaceutically acceptable salt thereof,

wherein B is cytosine or 5-fluorocytosine and R is selected from the group comprising H, monophosphate, diphosphate, triphosphate, carbonyl substituted with a C₁₋₆ alkyl, C₂₋₆ alkenyl, C₂₋₆ alkynyl, C₆₋₁₀ aryl, and

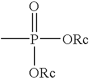

wherein each Rc is independently selected from the group comprising H, C₁₋₆ alkyl, C₂₋₆ alkenyl, C₂₋₆ alkynyl and a hydroxy protecting group.

In another embodiment, there is provided a method of treating a patient having cancer selected from the group comprising lung cancer, prostate cancer, bladder cancer, colorectal cancer, pancreatic cancer, gastric cancer, breast cancer, ovarian cancer, soft tissue sarcoma, osteosarcoma, hepatocellular carcinoma, leukemia and lymphomas in patients.

In another embodiment, there is provided a method of treating a patient having cancer selected from the group comprising colorectal cancer, melanoma, gastric cancer, islet cell cancer of the pancreas, non-small cell lung cancer (NSCLC), renal cancer, cervical cancer, breast cancer, ovarian cancer, squamous cell cancer of the pelvis, liver cancer, abdominal cancer and penile cancer.

In one embodiment, there is provided a method of treating non small cell lung cancer (NSCLC).

In another embodiment, there is provided a method of treating multi-drug resistant cancer.

In one embodiment, there is provided a method of treating cancer in a patient administering to said patient a therapeutically effective amount of cisplatin and a compound having formula I wherein the compound of formula I is β-L-OddC.

In one embodiment, there is provided a method for treating cancer in a patient comprising administering to said patient a therapeutically effective amount of cisplatin and at least one compound according to formula I and at least one further therapeutic agent chosen from the group comprising chemotherapeutic agent, multi-drug resistance reversing agent and biological response modifier.

In one embodiment, there is provided a method for treating cancer wherein the further therapeutic agent is a chemotherapeutic agent.

In one embodiment, there is provided a method for treating cancer wherein the further therapeutic agent is a chemotherapeutic agent chosen from Asparaginase, Bleomycin, Busulfan, Carmustine, Chlorambucil, Cladribine, Cyclophosphamide, Cytarabine, Dacarbazine, Daunorubicin, Doxorubicin, Etoposide, Fludarabine, Gemcitabine, Hydroxyurea, Idarubicin, Ifosfamide, Lomustine, Mechlorethamine, Melphalan, Mercaptopurine, Methotrexate, Mitomycin, Mitoxantrone, Pentostatin, Procarbazine, 6-Thioguanine, Topotecan, Vinblastine, Vincristine, Dexamethasone, Retinoic acid and Prednisone.

In another embodiment, there is provided a method for treating cancer wherein the further therapeutic agent is a chemotherapeutic agent chosen from Cytarabine, Etoposide, Mitoxantron, Cyclophosphamide, Retinoic acid, Daunorubicin, Doxorubicin and Idarubicin.

In another embodiment, there is provided a method for treating cancer wherein the further therapeutic agent is Doxorubicin.

In one embodiment, there is provided a method for treating cancer wherein the further therapeutic agent is a multi-drug resistance reversing agent.

In one embodiment, there is provided a method for treating cancer wherein the further therapeutic agent is PSC 833.

In one embodiment, there is provided a method for treating cancer wherein the further therapeutic agent is a biological response modifier.

In one embodiment, there is provided a method for treating cancer wherein the further therapeutic agent is a biological response modifier chosen from monoclonal antibodies and cytokines.

In another embodiment, there is provided a method for treating cancer wherein the further therapeutic agent is a cytokine chosen from interferons, interleukins and colony-stimulating factors.

In another embodiment, there is provided a method for treating cancer wherein the further therapeutic agent is a biological response modifier chosen from Rituxan, CMA-676, Interferon-alpha recombinant, Interleukin-2, Interleukin-3, Erythropoetin, Epoetin, G-CSF, GM-CSF, Filgrastim, Sargramostim and Thrombopoietin.

There is also provided pharmaceutically acceptable salts of the compounds of formula I of the present invention. By the term pharmaceutically acceptable salts of the compounds of formula (I) are meant those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toluene-p-sulphonic, tartaric, acetic, citric, methanesulphonic, formic, benzoic, malonic, naphthalene-2-sulphonic and benzenesulphonic acids.

Salts derived from appropriate bases include alkali metal (e.g. sodium), alkaline earth metal (e.g. magnesium), ammonium and NR₄+ (where R is C₁₋₄ alkyl) salts.

References hereinafter to the pharmaceutical combinations according to the invention includes compounds of the general formula I or a pharmaceutically acceptable salt thereof. Unless otherwise defined, all technical and scientific terms used herein have the same meaning as commonly understood by one of ordinary skill in the art to which this invention belongs. As used in this application, the term “alkyl” represents an unsubstituted or substituted (by a halogen, nitro, CONH₂, COOH, O—C₁₋₆ alkyl, O—C₂₋₆ alkenyl, O—C₂₋₆ alkynyl, hydroxyl, amino, or COOQ, wherein Q is C₁₋₆ alkyl; C₂₋₆ alkenyl; C₂₋₆ alkynyl) straight chain, branched chain or cyclic hydrocarbon moiety (e.g. methyl, ethyl, n-propyl, isopropyl, butyl, pentyl, hexyl, fluorohexyl, cyclopropyl, cyclobutyl, cyclopentyl, or cyclohexyl). The term alkyl is also meant to include alkyls in which one or more hydrogen atoms is replaced by an halogen, more preferably, the halogen is fluoro (e.g. CF₃— or CF₃CH₂—)

The terms “alkenyl” and “alkynyl” represent an alkyl containing at least one unsaturated group (e.g., vinyl, 1-propenyl, allyl, 1-methylpropenyl, 2-butenyl, 2-butenyl, ethynyl, 1-propynyl, or 2-propynyl).

The term “hydroxy protecting group” is well known in the field of organic chemistry. Such protecting groups may be found in T. Greene, Protective Groups In Organic Synthesis, (John Wiley & Sons, 1981). Example of hydroxy protecting groups include but are not limited to acetyl-2-thioethyl ester, pivaloyloxymethyl ester and isopropyloxycarbonyloxymethyl ester.

The term “aryl” represent an unsaturated carbocyclic moiety (e.g., phenyl and naphthyl), optionally mono- or di-substituted with OH, SH, amino, halogen or C₁₋₆ alkyl, and optionally substituted by at least one heteroatom (e.g. N, O, or S).

The term “multi-drug resistant cancer” represent a cancer tumour which is non responsive to treatment with chemotherapeutic agents.

The term “patient” represent any diseased human.

According to one embodiment, it will be appreciated that the amount of a compound of formula I of the present invention required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.01 to about 750 mg/kg of body weight per day, preferably in the range of 0.5 to 60 mg/kg/day, most preferably in the range of 1 to 20 mg/kg/day.

In another aspect, cisplatin and β-L-OddC are administered in one twenty four hour period at intervals of every two to five weeks. In another embodiment, cisplatin and β-L-OddC are adminstered consecutively at intervals of every two to five weeks.

The desired dose according to one embodiment is conveniently presented in a single dose or as divided dose administered at appropriate intervals, for example as two, three, four or more doses per day.

In another embodiment, the compound is conveniently administered in unit dosage form; for example containing 10 to 1500 mg, conveniently 20 to 1000 mg, most conveniently 50 to 700 mg of active ingredient per unit dosage form.

According to another embodiment of the present invention, the active ingredient is administered to achieve peak plasma concentrations of the active compound of from about 1 to about 75 μM, preferably about 2 to 50 μM, most preferably about 3 to about 30 μM. This may be achieved, for example, by the intravenous injection of a 0.1 to 5% solution of the active ingredient, optionally in saline, or orally administered as a bolus containing about 1 to about 500 mg of the active ingredient. Desirable blood levels may be maintained by a continuous infusion to provide about 0.01 to about 5.0 mg/kg/hour or by intermittent infusions containing about 0.4 to about 15 mg/kg of the active ingredient.

While it is possible that, for use in therapy, a compound of formula I of the present invention may be administered as the raw chemical, it is preferable according to one embodiment of the invention, to present the active ingredient as a pharmaceutical formulation. The embodiment of the invention thus further provides a pharmaceutical formulation comprising a compound of formula (I) or a pharmaceutically acceptable salt thereof together with one or more pharmaceutically acceptable carriers therefor and, optionally, other therapeutic and/or prophylactic ingredients. The carrier(s) must be “acceptable” in the sense of being compatible with the other ingredients of the formulation and not deleterious to the recipient thereof.

According to one embodiment of the present invention, pharmaceutical formulations include but are not limited to those suitable for oral, rectal, nasal, topical (including buccal and sub-lingual), transdermal, vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods according to this embodiment include the step of bringing into association the active compound with liquid carriers or finely divided solid carriers or both and then, if necessary, shaping the product into the desired formulation.

According to another embodiment, pharmaceutical formulation suitable for oral administration are conveniently presented as discrete units such as capsules, cachets or tablets each containing a predetermined amount of the active ingredient; as a powder or granules. In another embodiment, the formulation is presented as a solution, a suspension or as an emulsion. Still in another embodiment, the active ingredient is presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds of formula I according to an embodiment of the present invention are formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing an/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

For topical administration to the epidermis, the compounds of formula I, according to one embodiment of the present invention, are formulated as ointments, creams or lotions, or as a transdermal patch. Such transdermal patches may contain penetration enhancers such as linalool, carvacrol, thymol, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Formulations suitable for topical administration in the mouth include lozenges comprising active ingredient in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical formulations suitable for rectal administration wherein the carrier is a solid. In another embodiment, they are presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

According to one embodiment, the formulations suitable for vaginal administration are presented as pessaries, tampons, creams, gels, pastes, foams or sprays containing in addition to the active ingredient such carriers as are known in the art to be appropriate.

For intra-nasal administration the compounds, in one embodiment of the invention, are used as a liquid spray or dispersible powder or in the form of drops. Drops may be formulated with an aqueous or non-aqueous base also comprising one more dispersing agents, solubilising agents or suspending agents. Liquid sprays are conveniently delivered from pressurized packs.

For administration by inhalation the compounds, according to one embodiment of the invention are conveniently delivered from an insufflator, nebulizer or a pressurized pack or other convenient means of delivering an aerosol spray. In another embodiment, pressurized packs comprise a suitable propellant such as dichlorodifluoromethane, trichlorofluoromethane, dichlorotetrafluoroethane, carbon dioxide or other suitable gas. In another embodiment, the dosage unit in the pressurized aerosol is determined by providing a valve to deliver a metered amount.

Alternatively, in another embodiment, for administration by inhalation or insufflation, the compounds of formula I according to the present invention are in the form of a dry powder composition, for example a powder mix of the compound and a suitable powder base such as lactose or starch. In another embodiment, the powder composition is presented in unit dosage form in, for example, capsules or cartridges or e.g. gelatin or blister packs from which the powder may be administered with the aid of an inhalator or insufflator.

In one embodiment, the above described formulations are adapted to give sustained release of the active ingredient.

In one embodiment of the present invention, when the cisplatin and a compound of formula I or a pharmaceutically acceptable salt thereof is used in combination with a second therapeutic agent, the dose of each compound is either the same as or different from that when the compound is used alone.

In another embodiment, cisplatin is administered to the patient at a dose between about 20 mg/m² and about 100 mg/m² and at least one compound of formula I is administered to a patient at a dose between about 2.0 mg/m² and about 10 mg/m².

In another embodiment, cisplatin is administered to the patient at a dose between about 30 mg/m² and about 60 mg/m² and at least one compound of formula I is administered to a patient at a dose between about 2.0 mg/m² and about 10 mg/m².

In another embodiment, cisplatin is administered to the patient at a dose between about 60 mg/m² and about 90 mg/m² and at least one compound of formula I is administered to a patient at a dose between about 2.0 mg/m² and about 10 mg/m².

In another embodiment, cisplatin is administered to the patient at a dose between about 40 mg/m² and about 80 mg/m² and at least one compound of formula I is administered to a patient at a dose between about 4.0 mg/m² and about 7.0 mg/m².

In another embodiment, cisplatin and β-L-OddC are administered in one twenty four hour period at intervals of every two to five weeks. In another embodiment, cisplatin and β-L-OddC are administered consecutively at intervals of every two to five weeks.

In another embodiment, cisplatin and β-L-OddC are administered in one twenty four hour period at intervals of every three to four weeks. In another embodiment, cisplatin and β-L-OddC are administered consecutively at intervals of every three to four weeks.

The compounds of formula I as well as the use of the compounds of the present invention can be prepared according to the following examples which are provided to illustrate various embodiments of the present invention and shall not be considered as limiting in scope.

The entire disclosures of all applications, patents and publications, cited above and below, are hereby incorporated by reference.

### EXAMPLE 1

### Preparation of β-L-OddC

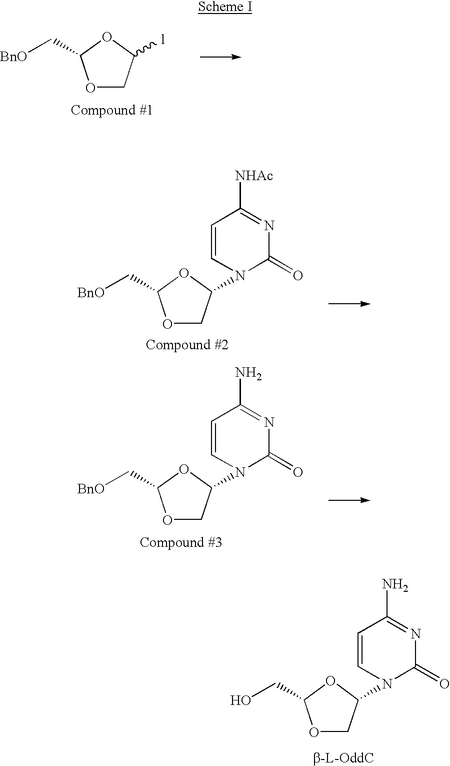

Compound #1:2S-Benzyloxymethyl-4R-iodo-1,3 Dioxolane and 2SBenzyloxymethyl-4S-iodo-1,3 dioxolane.

A mixture consisting of 2S-benzyloxymethyl-4S acetoxy-1,3 dioxolane and 2S-benzyloxymethyl-4R-acetoxy-1,3 dioxolane in 1:2 ratio (6 g; 23.8 mmol) was dried by azeotropic distillation with toluene in vacuo. After removal of toluene, the residual oil was dissolved in dry dichloromethane (60 ml) and iodotrimethylsilane (3.55 ml; 1.05 eq) was added at −78° C., under vigorous stirring. The dry-ice/acetone bath was removed after addition and the mixture was allowed to warm up to room temperature (15 min.). The ¹H NMR indicated the formation of 2S-benzyloxymethyl-4R-iodo-1,3-dioxolane and 2S-benzyloxymethyl-4S-iodo-1,3 dioxolane. ¹H NMR (300 MHz, CDCl₃) δ 3.65-4.25 (2H, m); 4.50-4.75 (4H, m) 5.40-5.55 (1H, overlapping triplets); 6.60-6.85 (1H, d of d); 7.20-7.32 (5H, m).

Compound #2: β-L-5′-Benzyl-2′-deoxy-3′-oxa-N-4-acetyl-cytidine

The previously prepared iodo intermediate (Compound #1) in dichloromethane, was cooled down to −78° C. Persylilated N-acetyl cytosine (1.1 eq) formed by reflux in 1,1,1,3,3,3-hexamethyl disilazane (HMDS) and ammonium sulphate followed by evaporation of HMDS was dissolved in 30 ml of dichloromethane and was added to the iodo intermediate. The reaction mixture was maintained at −78° C. for 1.5 hours then poured onto aqueous sodium bicarbonate and extracted with dichloromethane (2×25 ml).

The organic phase was dried over sodium sulphate, the solid was removed by filtration and the solvent was evaporated in vacuo to produce 8.1 g of a crude mixture. Based on ¹H NMR analysis, the β-L-5′-benzyl-2′-deoxy-3′-oxacytidine and its α-L isomer were formed in a ratio of 5:1 respectively. This crude mixture was separated by chromatography on silica-gel (5% MeOH in EtOAc) to generate the pure β-L (cis) isomer (4.48 g). Alternatively, recrystallization of the mixture from ethanol produces 4.92 g of pure β isomer and 3.18 g of a mixture of β and α-isomers in a ratio of 1:1. ¹H NMR (300 MHz, CDCl₃) δ 2.20 (3H, S, Ac); 3.87 (2H, m, H-5′), 4.25 (2H, m, H-2′); 4.65 (2H, dd, OCH₂Ph); 5.18 (1H, t, H-4′); 6.23 (1H, m, H-1′); 7.12 (1H, d, H-5); 7.30-7.50 (5H, m, Ph); 8.45 (2H, m, NH+H-6).

Compound #3: β-L-5′-Benzyloxy-2′-deoxy-3′-oxacytidine

The protected β-L isomer (4.4 g) (Compound #2) was suspended in saturated methanolic ammonia (250 ml) and stirred at room temperature for 18 hours in a closed vessel. The solvents were then removed in vacuo to afford the deacetylated nucleoside in pure form.

¹H NMR (300 MHz, CDCl₃) δ 3.85 (2H, m, H-5′); 4.20 (2H, m, H-2′); 4.65 (2H, dd, OCH₂Ph); 5.18 (1H, t, H-4′); 5.43 (1H, d, H-5); 5.50-5.90 (2H, br.S, NH₂); 6.28 (1H, m, H-1′); 7.35-7.45 (5H, m, Ph); 7.95 (1H, d, H-6).

Compound #4 β-L-OddC

β-L-5′-Benzyl-2′-deoxy-3′-oxacytidine (Compound #3) was dissolved in EtOH (200 ml) followed by addition of cyclohexene (6 ml) and palladium oxide (0.8 g). The reaction mixture was refluxed for 7 hours then it was cooled and filtered to remove solids. The solvents were removed from the filtrate by vacuum distillation. The crude product was purified by flash chromatography on silica-gel (5% MeOH in EtOAc) to yield a white solid (β-L-OddC)(2.33 g; 86% overall yield, α_{D}²²=−46.7° (c=0.285; MeOH) m.p.=192-194° C.). ¹H NMR (300 MHz, DMSO-d₆) δ 3.63 (2H, dd, H-5′); 4.06 (2H, m, H-2′); 4.92 (1H, t, H-4′); 5.14 (1H, t, OH); 5.70 (1H, d, H-5); 6.16 (2H, dd, H-1′); 7.11-7.20 (2H, brS, NH₂); 7.80 (1H, d, H-6) ¹³C NMR (75 MHz, DMSO-d₆) δ 59.5 (C-2′); 70.72 (C-5′); 81.34 (C-4′); 93.49 (C-1′); 104.49 (C-5); 140.35 (C-4); 156.12 (C-6); 165.43 (C-2).

### Example 2

### Evaluation of β-L-OddC and Cisplatin in Cancer Patients

A study was designed to determine the maximum tolerated dose of β-L-OddC and cisplatin. The patients selected were adult patients with solid tumours that were refractory to standard therapies. They had an ECOG performance status of between 0 and 2. They had adequate bone marrow, renal and hepatic function. There were 33 patients treated. Sixteen were male patients and seventeen were female. Twenty patients had colorectal cancer. Two patients had melanoma. One patient each had gastric cancer, islet cell cancer of the pancreas, non-small lung cancer (NSCLC), renal cancer, cervical cancer, breast cancer, ovarian cancer, squamous cell cancer of the pelvis, liver cancer, abdominal cancer and penile cancer. The dose escalation scheme is represented in Table 1. The terms LP and HP refer to lightly pre-treated and heavily pre-treated patient status. The term DLT refers to Dose limiting Toxicity.

TABLE 1

| | | Prior | Patients | | |
|---|---|---|---|---|---|
| β-L- | | Treat- | Started | | |
| oddC | Cisplat | ment | at this | Number | |
| Dose | in Dose | Status | dose | of | |
| (mg/m²) | (mg/m²) | (LP/HP) | level | Courses | Patients with DLT |
| 4.8 | 50 | 1/6 | 7 | 27 | 1 HP with prolonged |
| | | | | | neutropenia |
| 6.4 | 50 | 6/0 | 6 | 16 | 1 LP with prolonged |
| | | | | | neutropenia |
| 6.4 | 75 | 4/5 | 9 | 16 | 1 HP with prolonged |
| | | | | | neutropenia |
| | | | | | 1 HP with |
| | | | | | thrombocytopenia |
| 8 | 75 | 7/0 | 7 | 16 | 2 HP with febrile |
| | | | | | neutropenia |
| 0.75 | 50 | 0/4 | 4 | 7 | 1 HP with prolonged |
| daily × | | | | | neutropenia |
| 5 | | | | | |

As a result of the foregoing trial, it was discovered that one patient with metastatic non-small lung cancer (NSCLC) had a 42% reduction in disease extent after 2 courses of β-L-OddC/cisplatin. Best responses so far include six patients with stable disease, 21 with progressive disease and six still unknown. The recommended dose for heavily pre-treated patients is β-L-OddC 4.8 mg/m² and cisplatin 50 mg/m² administered every four weeks. The recommended dose for lightly pre-treated patients has not yet been determined.

The preceding examples can be repeated with similar success by substituting the generically or specifically described reactants and/or operating conditions of this invention for those used in the preceding examples.

While the invention has been illustrated with respect to the production and of particular compounds, it is apparent that variations and modifications of the invention can be made without departing from the spirit or scope of the invention.

We claim:

1. A pharmaceutical combination useful for the treatment of cancer in a mammal comprising a therapeutically effective amount of cisplatin and a compound having formula I: 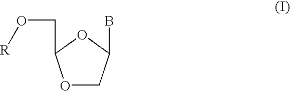
or a pharmaceutically acceptable salt thereof,
wherein B is cytosine or 5-fluorocytosine and R is selected from the group comprising H, monophosphate, diphosphate, triphosphate, carbonyl substituted with a C₁₋₆ alkyl, C₂₋₆ alkenyl, C₂₋₆ alkynyl, C₆₋₁₀ aryl, and 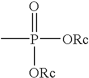
wherein each Rc is independently selected from the group comprising H, C₁₋₆ alkyl, C₂₋₆ alkenyl, C₂₋₆ alkynyl and a hydroxy protecting group.

2. The pharmaceutical combination according to claim 1 wherein R is H.

3. The pharmaceutical combination according to claim 1 wherein B is cytosine.

4. The pharmaceutical combination according to claim 1 wherein B is 5-fluorocytosine.

5. The pharmaceutical combination according to claim 1 wherein a compound of formula I is (−)-β-L-Dioxolane-Cytidine (β-L-OddC).

6. The pharmaceutical combination according to claim 1 wherein a compound of formula I is (−)-β-Dioxolane-5-fluoro-Cytidine (5-FddC).

7. The pharmaceutical combination according to claim 1 wherein the compound of formula I is substantially in the form of the (−) enantiomer.

8. The pharmaceutical combination according to claim 1 wherein the compounds of formula I of the present invention are at least 95% free of the corresponding (+) enantiomer.

9. The pharmaceutical combination according to claim 1 wherein the compounds of formula I of the present invention are at least 97% free of the corresponding (+) enantiomer.

10. The pharmaceutical combination according to claim 1 wherein the compounds of formula I of the present invention are at least 99% free of the corresponding (+) enantiomer.

11. The pharmaceutical combination according to claim 1 comprising a therapeutically effective amount of cisplatin and a compound of formula I wherein the compound of formula I is β-L-OddC.

12. The pharmaceutical combination according to claim 1 for use in the treatment of non small cell lung cancer.

13. The pharmaceutical combination according to claim 1 for use in the treatment of multi-drug resistant cancer.

14. A pharmaceutical combination according to claim 1 further comprising at least one further therapeutic agent chosen from the group comprising chemotherapeutic agent, multi-drug resistance reversing agent and biological response modifier.

15. A pharmaceutical combination according to claim 14 wherein the further therapeutic agent is a chemotherapeutic agent.

16. A pharmaceutical combination according to claim 14 wherein the further therapeutic agent is a chemotherapeutic agent chosen from Cytarabine, Etoposide, Mitoxantron, Cyclophosphamide, Retinoic acid, Daunorubicin, Doxorubicin and Idarubicin.

17. A pharmaceutical combination according to claim 14 wherein the further therapeutic agent is Doxorubicin.

18. A pharmaceutical combination according to claim 14 wherein the further therapeutic agent is a multi-drug resistance reversing agent.

19. A pharmaceutical combination according to claim 14 wherein the further therapeutic agent is PSC 833.

20. A pharmaceutical combination according to claim 14 wherein the further therapeutic agent is a biological response modifier.

21. The pharmaceutical combination according to claim 14 wherein the compound of formula I is β-L-OddC.

22. A method for treating a patient having cancer comprising
administering to said patient a therapeutically effective amount of cisplatin and a compound having the formula I: 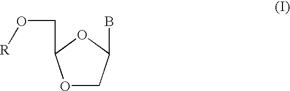
or a pharmaceutically acceptable salt thereof,
wherein B is cytosine or 5-fluorocytosine and R is selected from the group comprising H, monophosphate, diphosphate, triphosphate, carbonyl substituted with a C₁₋₆ alkyl, C₂₋₆ alkenyl, C₂₋₆ alkynyl, C₆₋₁₀ aryl, and 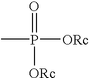
wherein each Rc is independently selected from the group comprising H, C₁₋₆ alkyl, C₂₋₆ alkenyl, C₂₋₆ alkynyl and a hydroxy protecting group.

23. The method according to claim 22 wherein the step of administering comprises administering to a patient with non small cell lung cancer.

24. The method according to claim 22 wherein the step of administering comprises administering to a patient with multi-drug resistant cancer.

25. The method according to claim 22 wherein said patient is administered a therapeutically effective amount of β-L-OddC and Cisplatin.

26. The method according to claim 22 wherein the compounds of formula (I) and the other therapeutic agents are administered to the mammal in need thereof sequentially.

27. The method according to claim 22 wherein the compounds of formula (I) and the other therapeutic agents are administered to the mammal in need thereof simultaneously.

28. A pharmaceutical formulation comprising a pharmaceutical combination according to claim 1 and least one pharmaceutically acceptable carrier or excipient.

29. A pharmaceutical formulation comprising a pharmaceutical combination according to claim 14 and least one pharmaceutically acceptable carrier or excipient.
